(19) United States
(12) Patent Application Publication
(10) Pub. No.: US 2025/0107756 A1
(43) Pub. Date: Apr. 3, 2025

(54) LIMITED FIELD OF VIEW LOCALIZATION FOR MAGNETIC RESONANCE MEDICAL IMAGING

(71) Applicant: Siemens Healthineers AG, Forchheim (DE)

(72) Inventors: Boris Mailhe, Plainsboro, NJ (US); Stefan Popescu, Erlangen (DE); Dorin Comaniciu, Princeton, NJ (US)

(21) Appl. No.: 18/476,496

(22) Filed: Sep. 28, 2023

(51) Int. Cl. A61B 5/00; A61B 5/055

(52) U.S. Cl. CPC A61B 5/70; A61B 5/0037; A61B 5/0077; A61B 5/055; A61B 5/7267

(57) ABSTRACT

Rather than the whole-body scout scan, multiple scout scans of different regions are used to locate the target for magnetic resonance scanning. A diagnostic scan may be planned in as little time as possible based on localization using multiple scout scans of different regions of the patient. The planning of the different regions may be optimized to minimize the number and/or time for localizing the target.

### BACKGROUND

Magnetic resonance (MR) imaging generates a signal using radio-frequency (RF) pulses to excite spins (typically protons) at their Larmor frequency with respect to a constant strong magnetic field B0. A mismatch between the excitation frequency and field strength causes a loss of signal due to off-resonance. This is commonly used to perform spatially selective excitation: gradient coils generate spatial modulations of the main B0 field, and a narrowband RF excitation only generates signal within a given field isochromat instead. The gradient and RF systems can be jointly programed to target any spatial subregion within the overall scanning volume (field of view (FoV)). To perform a clinical scan, this programing requires prior knowledge of the location of the target patient region of interest within the scanning volume.

In current whole-body scanners, the main B0 and RF fields are built to be spatially homogeneous within a large spherical scanning volume. Additional shimming subsystems may be used to improve homogeneity. The gradients are built for a spatially linear response, allowing selection of slices, slabs, and cubes within the scanning volume. Localization and scan planning is performed in multiple steps. The patient table or other patient support component is mechanically moved so that the target anatomy roughly fits within the scanning volume (i.e., the FoV of the MR scanner). This can be controlled visually by the technologist or automated with the help of external sensors, such as cameras. A large FoV, low-resolution scan of the whole scanning volume, called a scout or localizer, is performed. The target anatomy is detected in the resulting image either manually or automatically. Diagnostic scans are planned by using the target anatomy position. The scan plan is converted into controls for the RF and gradients components for the diagnosis scan.

This workflow relies on the ability to acquire large FoV scout scans thanks to a large homogeneous volume. For a compact (e.g., organ-specific) MR system with a much smaller scanning volume, the initial mechanical alignment may fail to position the target anatomy within the scanning volume, especially if the target anatomy is deep within the body and hard to locate from surface information (e.g., the prostate).

### SUMMARY

By way of introduction, the preferred embodiments described below include methods, systems, instructions, and non-transitory computer readable media for localizing a target in MR scanning. Rather than the whole-body scout scan, multiple scout scans of different regions are used to locate the target. A diagnostic scan may be planned in as little time as possible based on localization using multiple scout scans of different regions of the patient. The planning of the different regions may be optimized to minimize the number of scout scans and/or time for localizing the target.

In a first aspect, a method is provided for localizing a target for MR scanning. An initial position of the target relative to a magnetic resonance scanner is estimated from first data. The target is an object in a patient. The first data is pre-scan information. Multiple scout scans are performed for different regions of the patient. The different regions are based on the initial position. A subsequent position of the target relative to the magnetic resonance scanner is determined from the scout scans. The target is diagnostically imaged with the MR scanner. The imaging is configured by the subsequent position of the target.

In a second aspect, a magnetic resonance (MR) system includes: a MR scanner configured by settings of controls to scan a diagnostic region of a patient, the scan providing scan data, the diagnostic region being within a homogenous volume field of view of the MR scanner; and a processor configured to scout scan different scout regions of the patient using values of the settings, configured to determine a location of the diagnostic region from the scout scan of the different scout regions, and configured to perform the scan of the diagnostic region based on the determined location.

In a third aspect, a method is provided for localizing a target for magnetic resonance (MR) scanning. A magnetic resonance system performs multiple scout scans of different regions of the patient. The magnetic resonance system has a field of view corresponding to a diameter of a spherical homogenous volume of 30 cm or less such that each of the different regions are outside the field of view of others of the different regions. A machine-learned model implemented by a processor determines a position of the target relative to the magnetic resonance system from the multiple scout scans. The target is imaged with the magnetic resonance system. The imaging is configured by the position of the target.

Further aspects are provided as illustrative examples below. Any of the illustrative examples may be used with different of the aspects above.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

FIG. 1 is a block diagram of an implementation of an MR system for localizing a target;

FIG. 2 is a flow chart diagram of one implementation of a method for localizing a target for MR scanning;

FIG. 3 is a flow chart diagram of another implementation of the method for localizing a target for MR scanning;

FIG. 4 illustrates examples of different scout regions relative to a target;

FIG. 5 illustrates different examples of different scout regions relative to a target; and

FIG. 6 illustrates yet other examples of different scout regions relative to a target.

### DETAILED DESCRIPTION

A flexible workflow is provided for localization of the target anatomy with MR scanners. The workflow may be used with whole-body MR scanners or MR scanners having a smaller extension of the FoV (e.g., <50 cm diameter of spherical homogenous volume (DSHV), or <30 cm DSHV, or <10 cm DSHV). The workflow may use scout scan regions of inhomogeneous static B0 field and gradient fields as those regions may be located outside the DSHV.

The workflow computes an initial estimate of the target anatomy location from pre-scan data, such as only from pre-scan data. Multiple scout scans are performed at different locations to cover a superset of the target anatomy. All the acquired scout scans and a first estimation of the target anatomy location are used to exactly locate the target anatomy and to plan the diagnosis scan.

As compared to the typical workflow for whole-body scanners, multiple scout scans are used because a single scan cannot guarantee that the target anatomy is covered by the scanned area. The sequence of scout scans itself is planned. Since multiple scout scans can be acquired, they may use different MR contrast weightings to focus on different anatomical landmarks and structures. Whole-body anatomical information may be leveraged from previous scans, if available, for the initial estimate. Localization and scan planning may be performed using not only MR subsystem controls but also mechanical controls to move the patient with respect to the scanning volume and/or to move the measuring system with respect to the target anatomy. Localization and scan planning artificial intelligence (AI) components extrapolate information outside of the currently acquired field of view for localizing the target.

These differences and/or the flexible workflow enable localization with inhomogeneous devices with a smaller scanning volume. The flexible workflow may also be used with whole-body scanners. Highly accelerated, non-imaging scout scans (e.g., radial lines or narrow blades) could be used to speed up localization in whole-body scanners. A succession of highly accelerated scout scans using multiple contrasts may improve the localization performance of some anatomical structures that have poor contrast in the currently performed scout scans. Whole-body systems equipped with parallel RF transmit can perform selective excitation of small volumes. This selective excitation of small volumes may be used for localization, which may be beneficial in some applications. Deeper integration of table motion with the localization and/or planning may improve clinical workflows, e.g., by placing the target anatomy closer to the scan isocenter and reducing geometrical distortions due to larger gradient inhomogeneities close to the boundary of the FoV of the scanner. Leveraging prior information from previous scans (“delta-scanning”) may reduce scan times and increase the clinical accuracy of scans in a longitudinal follow-up workflow.

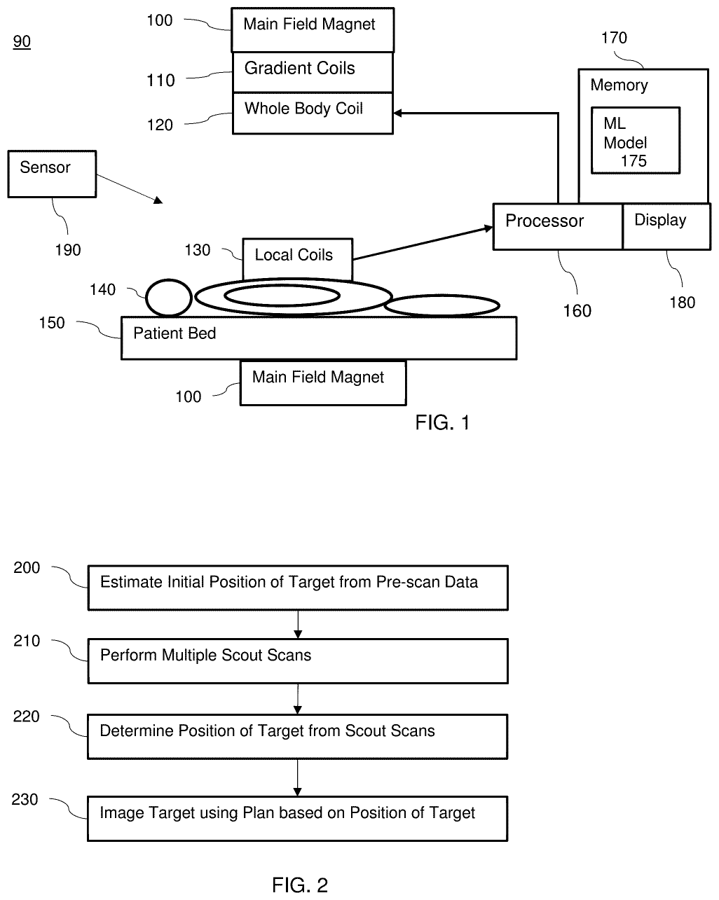

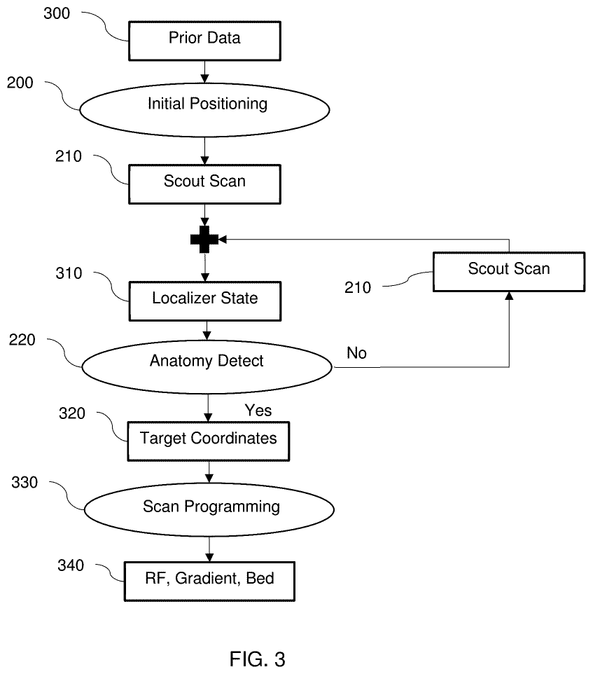

FIG. 1 shows one embodiment of a MR system for MR scanning by an MR scanner 90. The MR scanner 90 scans a given patient 140. The MR scanner 90 performs the acts of FIG. 2, FIG. 3, or another method. The MR scanner 90 localizes a target in the patient 140 based on multiple scout scans. The MR scanner 90 may have reduced requirements, such as non-homogenous fields and/or non-linear gradients. The MR scanner 90 may be capable of image reconstruction but may use more rapid or different scanning for direct determination of an analytic from scan data.

The MR scanner 90 includes a main field magnet 100, gradient coils 110, whole body coil 120, local coils 130, and a patient support (e.g., bed) 150. The system includes the MR scanner 90, processor 160, memory 170, and display 180. One or more sensors 190 separate from the coils 110, 120, 130 may be provided. Additional, different, or fewer components may be provided for the MR scanner 90 and/or system. For example, the local coils 130 or the whole-body coil 120 are not used. In another example, the processor 160, memory 170, and display 180 are provided without the coils 100-120 and patient support 150, such as a workstation operating on scan data stored in the memory 170. In yet another example, the processor 160, memory 170, and/or display 180 are part of the MR scanner 90.

The MR scanner 90 is configured by settings of controls to scan a region of the patient 140. The scan provides scan data in a scan domain. The MR scanner 90 scans the patient 140 to provide raw measurements (measurements in a possibly non-linear frequency domain). Where hardware imperfections make the spatial encoding non-Fourier, the measured responses are referred to as raw data or scan data rather than k-space data. Where spatial encoding is Fourier, the scan or raw data may be k-space data. For the scan, the main field magnet 100 creates a static base magnetic field, B0, in the body or part of the body of the patient 140 positioned on the patient support 150. The gradient coils 110 produce position dependent magnetic field gradients superimposed on the static magnetic field. The gradient coils 110 produce position dependent and shimmed magnetic field gradients in three orthogonal directions and generate magnetic field pulse sequences. The whole-body coil 120 and/or the local coils 130 receive radio frequency (RF) transmit pulses, producing magnetic field pulses (B1) that rotate the spins of the protons in the imaged region of the patient 140.

In response to applied RF pulse signals, the whole-body coil 120 and/or local coils 130 receive MR signals, i.e., signals from the excited protons within the body as they return to an equilibrium position established by the static and gradient magnetic fields. The MR signals are detected and processed by a detector, providing an MR dataset of raw data. A raw storage array of the memory 170 stores corresponding individual measurements forming the MR dataset.

The MR scanner 90 is configured by the processor 160 to scan. Any of various scanner controls may be set, such as k-space coordinates, TR, TE, flip angle, pulse envelopes, carrier frequencies, timings, durations, and/or raw transmit pulses. A protocol, with or without user input or alteration, may establish the settings, at least initially, used for a particular scan. Any level of generality may be provided for the settings, such as an abstraction of the actual variables used for specific hardware. The memory 170 stores the configuration (e.g., a predetermined pulse sequence of an imaging protocol and a magnetic field gradient and strength data as well as data indicating timing, orientation, and spatial volume of gradient magnetic fields to be applied in scanning) and the resulting raw data or measurements.

This scan plan relies, in part, on the location of the target. The spatial positioning of the scan is located to scan the target. This target location is within a homogenous volume FoV of the MR scanner 90 by the scan plan. By locating the target, the most homogenous FoV of the MR scanner 90 may be positioned at the target for the diagnostic scan.

The MR scanner 90 may be an image caliber MR scanner 90, such as having a homogenous B0 field provided by 0.5 T or higher field strength. An imaging caliber B0 field has, for example, <0.5 ppm VRMS over the volume of interest (e.g., a spherical volume with a diameter greater than 50 cm). The image caliber MR scanner 90 provides imaging caliber linear gradients, which have, for example, <2% geometric distortion.

In other embodiments, the MR scanner 90 has less restrictive design constraints, such as being designed and built for analytics without reconstruction and/or use for imaging small regions. For example, a non-uniform main magnetic field (e.g., 10% variation in scan region of patient), non-homogeneous B0 field or transmit pulses (e.g., >0.5 ppm), and/or non-linear gradients (e.g., >2% geometric distortion) are provided. As another example, the main magnet 100 is 0.1 T or less. Where this less uniform magnetic field is used, the bore in which the patient lies during scanning may be open. An open bore scanner allows sufficient space that a field of view of the sensor 190 may sense (e.g., take a picture or see) the patient within the bore. The field of view of the sensor 190 extends to the patient while the patient is within the open bore of the MR scanner 90. For example, the open bore may include a chair or bed as the patient support 150 without a surrounding housing or may be a housing that is open at more than the two ends along a longitudinal direction of the patient 140, such as having open sides and open ends (i.e., housing above and below the patient but not to the top, bottom, or sides). As another example, the bore may be cylindrical, but over 4, 5, or 6 feet in diameter.

For a less restrictive design, the MR scanner 90 may be in a room without a faraday cage. Radio frequency shielding is not provided outside of or surrounding the MR scanner 90. In other embodiments, the MR scanner 90 is in a room formed as a faraday cage.

For a less restrictive design, the homogenous region of the B0 field may be smaller, such as less than 50 cm, less than 30 cm, or even less than 10 cm. While <0.5 ppm VRMS is provided for this smaller FoV, the FoV does not cover a majority of the patient while positioned on the patient bed 150. This homogenous region is smaller than a typical patient or scanner sized for a whole-body of a patient.

The patient support 150 is a flat or contoured slab (e.g., bed) on which the patient 140 lies or is supported. In an open bore, the patient support 150 may be formed as a recliner or chair given a larger bore.

The patient support 150 is movable relative to the MR scanner 90 (i.e., the main field magnet 100, gradient coils 110, and whole-body coil 120). A motor (actuator) with gearing, pulleys, and/or other transmission moves the patient support 150 into and out of the bore, such as longitudinally along the bore or patient support 150. Other motion may be provided, such as raising and lowering the patient support 150, moving the patient support 150 laterally (orthogonal to a side of the patient 140 lying on their back), and/or rotating along one, two, or three dimensions. One or more sensors may measure the location of the patient support 150 relative to the MR scanner 90.

The patient support 150 with the patient 140 is moved into the more or most homogeneous part of the magnetic field created by the main field magnet. Using the gradient coils 110, the MR scanner 90 may localize a region of interest or scan region at different locations in a field of view (FoV) of the MR scanner 90. The patient support 150 moves the patient 140 so that the region of interest is within the FoV of the MR scanner 90 to allow localization. For example, the prostate is to be scanned. The patient support 150 moves the lower abdomen of the patient 140 to be centered in the bore and/or within the bore. The MR scanner 90 then performs multiple scout scans. The scout scans are of different regions of the patient. The patient support 150 may move and/or the frequency of the RF pulses may be altered to scan the different regions for localization. In alternative embodiments, the open bore allows the patient to move themselves within the bore or MR scanner 90 FoV.

The sensor 190 is one or more sensors. The sensor 190 is positioned outside of the bore or MR scanner 90 FoV but may be within the bore. The sensor 190 mounts to the housing of the MR scanner 90, a robotic arm, wall, ceiling, or sensor tree. The sensor 190 is positioned so that the sensor FoV captures all or part of the patient 140 while the patient 140 is within the bore. For example, the sensor FoV reaches an exterior part of the patient 140 while the patient 140 is positioned on the patient support 150 where the MR scanner 90 may be localized to scan the diagnostic region of interest.

The sensor 190 is an active or passive sensor. For example, the sensor 190 is a camera for acquiring optical images or a depth camera for acquiring optical images with depth. Infrared cameras or cameras for visual frequencies may be used. Other types of passive sensors may be provided, such as laser rangefinders, radio frequency sensors, or a weight mat sensing weight at different locations on the patient support 150. In an alternative, or additional, example, the sensor 190 is an active sensor, such as an ultrasound scanner that transmits acoustic energy in a steerable way and receives echoes or a camera on a robotic arm that actively moves the camera.

The processor 160 configures the MR scanner 90. The processor 160 is a general processor, digital signal processor, graphics processing unit, application specific integrated circuit, field programmable gate array, artificial intelligence processor, tensor processor, digital circuit, analog circuit, combinations thereof, or another now known or later developed device for operating on raw data, localizing, controlling, and/or applying artificial intelligence. The processor 160 is a single device, a plurality of devices, or a network. For more than one device, parallel or sequential division of processing may be used. Different devices making up the image processor may perform different functions, such as configuring the MR scanner 90 to scan by one device and detecting a location of a target by another device. In one embodiment, the processor 160 is a control processor or other processor of the MR scanner 90. Other processors of the MR scanner 90 or external to the MR scanner 90 may be used.

The processor 160 is configured by software, firmware, and/or hardware to perform its tasks. The processor 160 operates pursuant to instructions stored on a non-transitory medium (e.g., memory 170) to perform various acts described herein.

The processor 160 is configured to control the MR scanner to scout scan different scout regions of the patient. By configuring the MR scanner 90 with different values of the settings, different regions of the patient 140 may be scanned to scout for the target in the patient relative to the MR scanner 90. The values are changed to scan the different regions. Other characteristics may be altered for different scout scans and corresponding regions, such as using different contrast weightings for the different scout regions.

The processor 160 may be configured to estimate an initial location of the diagnostic region. The estimate may be from a previous whole-body scan. The relationship of the target within the patient to the exterior of the patient may be determined from the previous whole-body scan, allowing positioning of the patient 140 relative to the MR scanner 90 as an initial estimate. Other sources of pre-scan data (information prior to the MR scout scans of a current imaging appointment) may be used for estimating an initial location of the target relative to the MR scanner 90, such as patient registration information (e.g., height and weight used to estimate location of internal anatomy relative to the exterior of the patient) or images from the sensor 190. Alternatively, an operator manually positions the patient (e.g., placing a part of the exterior of the patient at an isocenter of the FoV of the MR scanner 90).

Different scout regions are established based on the initial location. More than one region of the patient 140 is scanned by the MR scanner 90 to localize the position of the target (e.g., organ such as the prostrate) relative to the MR scanner 90. One or more of the scout regions may be positioned using the initial location. For example, the estimate of the initial location is located at or close to a center of the FoV of the MR scanner 90. One scout region is centered on the initial location. Other scout regions around that scout region may be selected or used. As another example, one scout region is positioned at a landmark with a known or expected relationship to the target. The scout region is scanned and used to then estimate the location of the target better, which is then scanned as another scout region.

The processor 160 is configured to cause performance of the scout scan and/or the scan of the diagnostic region with controlled movement of the patient support 150. For the scout scans with limited FoV, the patient support 150 may be moved to allow scanning of the different regions. Alternatively, or additionally, the patient 140 is moved relative to the patient support 150. In yet another alternative, the MR scanner 90 scans at different frequencies so has scout regions corresponding to different regions of homogeneity. For the diagnostic scan, the processor 160 may control the patient support 150 so that a target larger than the FoV may be entirely scanned with a sequence of diagnostic scans.

AI may be used to localize (estimate initial position from pre-scan data and/or detect the target from scout scan results) and/or determine the scout scan regions (e.g., the different regions). The AI may have been trained to extrapolate information outside of a current FoV, allowing the processor 160 to estimate where a next scout scan should occur. A search pattern or default pattern of different regions may alternatively be used.

In one implementation, the processor 160 applies the AI (e.g., a machine-learned model 175) to estimate the initial position of the target from pre-scan data, detect the target from the scout scans, and/or to control where to place different scout scan regions. The machine-learned model 175 may be a neural network with one or more inputs (e.g., pre-scan data and/or scout scan data) and outputs a target position and/or control of a next scout scan. The machine-learned model is trained to control the MR scanner to act on the patient support 150 and/or MR scanner 90.

The machine-learned model 175 is one or more models. Hierarchal, sequential, multi-task, or other machine-learned model arrangements may be used. The machine-learned model 175 is formed from one or more networks and/or another machine-learned architecture (e.g., support vector machine). For example, and used herein, the machine-learned network is a deep-learned neural network. In another example, the machine-learned network is a neural network of a sequence of transformer and/or attention layers. In one embodiment, the machine-learned model 175 includes models for detection. Different actions (e.g., instructions to move patient support 150 and/or configure MR scanner 90 for scanning a different scout region) may be performed by a machine-learned model (e.g., a reinforcement deep learned network or machine-trained actor) or different machine-learned models for the different actions.

The machine learned model 175 is trained by training data with or without ground truth. A loss based on output of the model being trained compared to an objective function or ground truth is used in an optimization to train the model. A reward may be used in training, such as rewarding more rapid localization. Any optimization may be used, such as Adam. Any loss may be used, such as cross entropy, L1 loss, or L2 loss. Pre-training, cross-training, and/or continuous training may be used. The training data is gathered from a database of examples performed under expert control. Ground truth may be curated or created by expert review. Instead, or in addition, the training data may be created by modeling or synthetically created using a model of MR scanning.

The processor 160, using the AI (e.g., machine-learned model 175), is configured to determine a location of the diagnostic region. The scout scans of the different scout regions are input or used detect the location of the target. Other detection than AI may be used.

The processor 160 is configured to cause the MR scanner 90 to perform the scan of the diagnostic region based on the determined location. The location is used to plan the diagnostic scan, such as establishing a position and/or orientation of the target. The diagnostic scan is performed where the target, as localized, is positioned in the FoV of the MR scanner 90.

The memory 170 is a cache, buffer, RAM, removable media, hard drive, or another computer readable storage medium. Computer readable storage media include various types of volatile and nonvolatile storage media.

The memory 170 stores raw data (e.g., scan data), the settings for the controls, pre-scan data, the machine-learned model 175, positions, localization, scan plans, protocol settings (values), and/or other data. The memory 170 may alternatively or additionally store instructions for the processor 160. The functions, acts or tasks illustrated in the figures or described herein are executed by the processor 160 in response to one or more sets of instructions stored in or on the non-transitory computer readable storage media of the memory 170. The functions, acts or tasks are independent of the particular type of instructions set, storage media, processor or processing strategy and may be performed by software, hardware, integrated circuits, firmware, micro code, and the like, operating alone or in combination.

In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the way the present embodiments are programmed.

The display 180 is a CRT, LCD, plasma, projector, printer, or other display device. The display 180 is configured by loading an image to a display plane or buffer. The display 180 is configured to display diagnostic images from diagnostic scanning of the target of the patient. Information from scout scans, such as an image showing detection of the target, may be displayed by the display 180. Where the MR scanner 90 is a low-cost scanner with no or poor imaging capability, the scan may be used as a first test with cheaper equipment to determine whether more costly MR or other type of medical imaging should be performed.

FIG. 2 is a flow chart diagram of one implementation of a method for localizing a target for MR scanning. FIG. 3 shows a flow chart diagram of another implementation of the method for localizing the target for MR scanning. In these methods, different scout regions are scanned, and the target for diagnostic imaging is located from the combination of scout scans. Low quality or small FoV MR scanners may localize the target relative to the MR scanner without a whole-body scan.

The methods are performed by the system of FIG. 1 or another system. A processor estimates in act 200. The MR scanner performs multiple scout scans in act 210. The processor determines the position of the target in act 220. The processor programs the MR scanner to scan the target in act 330, and the MR scanner images the target in a diagnostic scan based on the program or scan plan in act 230. Other components may be used, such as a remote server or a workstation performing the estimation of act 200, detection of act 220, and/or programming of act 330.

Various acts may use a machine-learned model (i.e., AI). For example, one machine-learned model is used for act 200, and another machine-learned model is used for act 220. During application of the machine-learned model to one or more different patients and corresponding different scan data, the same learned weights or values of the machine-learned model are used. The model and values for the learnable parameters are not changed from one patient to the next, at least over a given time (e.g., weeks, months, or years) or given number of uses (e.g., tens or hundreds). These fixed values and corresponding fixed model are applied sequentially and/or by different processors for different patients. The model may be updated, such as retrained, or replaced but does not learn new values as part of application for a given patient. In other embodiments, continuous learning is used.

The method is performed in the order shown (top to bottom or numerical) or other orders. Additional, different, or fewer acts may be provided. For example, act 200 is not provided, such as being replaced by manual positioning of the patient. As another example, act 230 is not provided, such as where the scan plan is created but not yet used or used for generating an analytical value.

In act 200, the processor estimates an initial position of the target relative to a MR scanner. The patient may be positioned relative to the scanner, but the alignment is not precise. The location of internal anatomy or object (e.g., target) is not know precisely as well. The processor estimates a registration or alignment of the target relative to the MR scanner to begin localization of the target relative to the MR scanner.

The processor estimates the initial position (e.g., location in 2 or 3 dimensions) from data. An object in the patient is estimated from pre-scan data 300. Data 300 acquired before a current MR scan (i.e., before the scout scans and before the diagnostic scan of an imaging appointment for the patient in the MR scanner). The pre-scan data 300 only is used to for an initial estimate. In other embodiments, the initial estimate is not made. Instead, the patient is positioned relative to the MR scanner based on exterior landmarks of the patient (e.g., placing a lower torso in the FoV for prostate localization).

One or more of different types of data are used to make the initial estimate. For example, patient registration data, measurements from an exterior of the patient, and/or a previously acquired representation of an interior of the patient is used. Patient registration data may include height, weight, pre-existing images of the patient acquired with their own picture or video cameras, or digital models of the patient internal anatomy computed from such images. This registration data may be used to estimate the location of the internal anatomy relative to the exterior of the patient, which exterior may be used to align or register in the FoV of the MR scanner. Measurements from external sensors attached to the scanning device or present in the scanning room include video cameras, depth cameras, laser or ultrasonic rangefinders, LiDAR. These measurements may be used to estimate the target location relative to the exterior of the patient and/or are used to register the exterior of the patient to the MR scanner. Other information (e.g., patient registration or previously acquired images) may be used to relate the internal target to the patient exterior. Prior information about the internal anatomy of the same patient may be in the form of previously acquired, large FoV tomographic images such as CT or MR images acquired with a whole-body scanner. Such images are available in the context of a longitudinal follow-up clinical workflow. Models computed from such images may be used instead of the images. These prior images are automatically recalled from the picture archiving and communications system (PACS) of the hospital or medical insurer.

The processor estimates. The estimate may be based on modeling. The model is fit to spatial information derived from the pre-scan data, relating the estimated location of the target to the MR scanner. In another approach, the processor applies AI. The AI estimates the initial position in response to input of the pre-scan information. The AI is trained to accept pre-scan data as input and compute the initial estimate of the target anatomy location. The AI may output uncertainty information about this estimate. Prior camera or tomographic images of the patient may be available or not depending on patient-specific clinical workflows. The AI system is implemented and trained to be robust to the presence or absence of pre-scan patient image or body model anatomy data. The location of the target relative to the exterior of the patient and the exterior of the patient relative to the MR system are estimated.

In act 210, the MR scanner performs multiple scout scans. The processor controls the MR scanner, so sets the values of settings for MR scanning in scout scans.

The multiple scans are of different regions of the patient. The FoV and/or scan region is moved relative to the patient to scan different regions of the patient. In one approach, the MR scanner has a limited FoV, such as a FoV with a diameter of spherical homogenous volume of less than 50, 30, and/or 10 centimeters. Using multiple scout scans of different regions provides a flexible workflow for localization of the target anatomy with any MR scanners having a smaller extension of the FoV (for example <50 cm diameter of spherical homogenous volume, or <30 cm DSHV, or <10 cm DSHV). One or more of the different scan regions may expand the FoV, such as scanning within scan regions of inhomogeneous static B0 field and gradient fields as regions located outside the DSHV.

The different regions overlap or do not overlap. For example, each of the different regions are outside the field of view of others of the different regions. As another example, part but not all of a region for scout scanning overlaps with part but not all of another region for scout scanning. Several scout scans at different locations cover a superset of the target anatomy not covered by the FoV of the scanner at a single location relative to the patient.

The initial location estimate is used to perform the multiple scout scans. For example, one of the regions is the FoV of the MR scanner with the initial estimate of the target location placed at or near a center of the FoV (e.g., near being within 5 cm). As another example, a sequence of scout scans to scan at different locations relative to the initial estimate of the target location are used. A default pattern may be provided for the scout scans. The initial location estimate of act 200 is used to plan and acquire one or multiple scout scans for detection of the target anatomy in the acquired scout scans. Each scout scan covers a field of view at another spatial location.

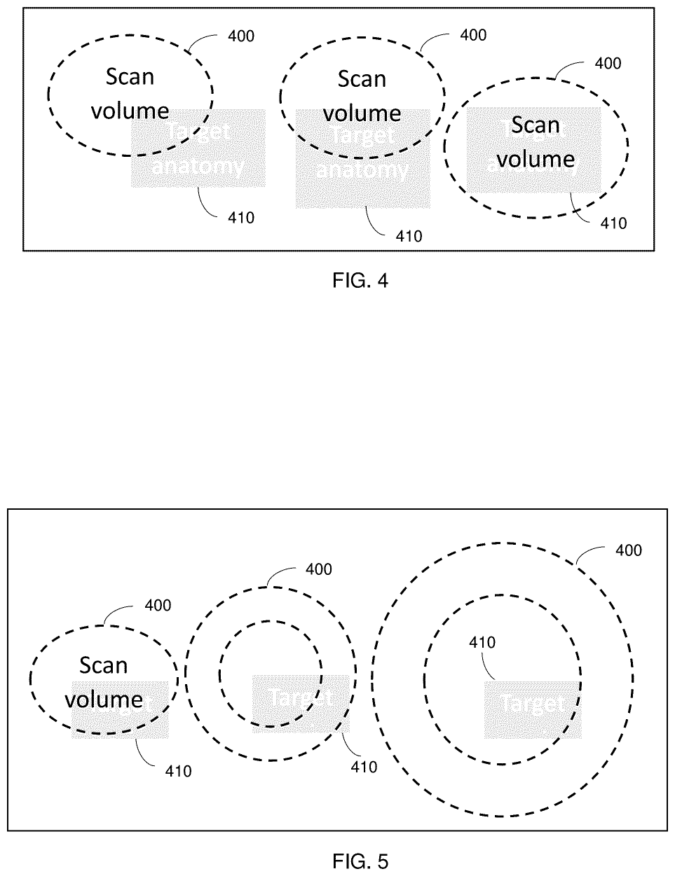

For scanning the different regions, the FoV may be controlled by mechanical movement of the patient and/or MR scanner or by frequency change of the MR scanner. In one approach, the patient is moved by robotics of the patient support. The patient is physically moved relative to the MR scanner by movement of the patient bed and/or the MR scanner. The MR scanner FoV relative to the patient shifts as part of this movement, allowing the MR scanner to perform scout scans at different regions of the patient. FIG. 4 shows an example. The FoV is the scan volume 400, which is shown relative to the target anatomy 410, such being placed based on the initial estimate. FIG. 4 shows shifting position of the patient, and thus the target anatomy 410, relative to the scan volume 400. Different scan volumes 400 are scout scanned.

In another approach, a central frequency of the RF pulses of the MR scanner is adjusted. One of the regions may be scanned using the Larmor frequency, so corresponds to the FoV of the MR scanner relative to the patient as initially positioned. By adjusting the frequency, other regions are scanned. The frequency offsets the scan from the homogenous region of the FoV to another region with a different B0 field strength so that at least some of the different regions of the scout scans are outside of a homogenous scanning volume. FIG. 5 shows an example. The different regions 400 correspond to the FoV (circle) and different rings about the FoV that are isochromatic up to a tolerance determined by the bandwidth of the pulse. The central frequency of the RF system is adjusted to generate and receive MR signals from outside of the homogeneous scanning volume for some of the regions (e.g., the rings of FIG. 5). By additionally applying image reconstruction algorithms able to deal with inhomogeneous spatially encoding fields, scout scan data for different regions is acquired.

The acquired scout scans with or without the initial estimation of the target anatomy location are then used to exactly locate the target anatomy in act 220 and to plan the diagnosis scan in act 230. In act 220, the processor determines a subsequent (e.g., actual) position of the target relative to the MR scanner. The scout scans are used to determine the position. For example, a machine-learned model implemented by a processor determines the position of the target relative to the MR system from the multiple scout scans.

Raw data, k-space data, other scan data, reconstructed images, or data derived therefrom may be used to detect the target in act 220. In some approaches, the scout scans are imaging scans acquired using k-space sampling. Reconstructed images are aggregated. In other approaches, the scout scans are highly accelerated, and the acquired raw measurements are used as the input.

In one approach, the scout scan data as images is aggregated. The position is estimated from the aggregation. Each scout scan only provides a partial observation of the patient anatomy. A localizer state aggregates all scout images to represent all currently available information covering a larger patient anatomy (i.e., larger than each individual scout image). In some implementations, the localizer state is the concatenation of the data acquired in each scout scan. For example, all scout scans are planned at the beginning of act 210 to acquire comprehensive spatial coverage of a region believed to contain the target anatomy with high probability according to the initial estimate. Then, an AI trained to accept all acquired scout scans as its input detects the location of the target anatomy.

In other implementations, an AI encoder is trained to transform each scout scan into a latent representation. The localizer state is the concatenation of the latent representations of all scout scans. The concatenated latent representations are used by the AI to detect the location of the target.

Another implementation is shown in FIG. 3. An AI aggregator is trained to update the localizer state 310 with each new scout scan 210. Where the localizer state 310 is a latent representation or image, the AI aggregator accepts the previous localizer state 310 and the latent representation of the new scout scan 210 as an input and outputs the updated localizer state 310 based, in part, on the information acquired with the last scout scan 210. The anatomy detection of act 220 is then performed for the updated or current localizer state 310.

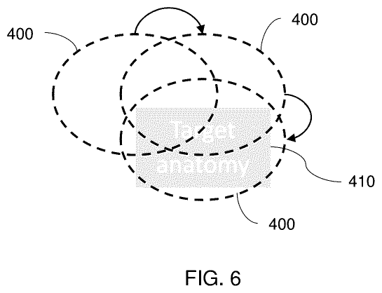

In one implementation, the performance of the scout scan in act 210 and the determination of the position through anatomy detection of act 220 are interleaved, such as shown in FIGS. 3 and 6. The AI is used to not just detect the target anatomy 410, but also to select the next scout scan region 400. For example, the AI is a deep reinforcement learning (DRL) AI or other action AI (trained actor). The DRL AI selects one or more different regions 400 based on the detection from the current localizer state 310 (based on previous ones of the scout scans with or without the initial estimate and/or pre-scan data). The scout scans are planned and acquired iteratively. The AI is trained to accept the initial estimate from act 200 and any previously acquired scout scans 210 as an input and output a plan for the next scout scan to acquire or to stop once the target anatomy has been detected. Using DRL, the AI outputs a value indicating a confidence in detection of the target. A policy of the AI outputs a decision for location of the next scout scan region 400 (see arrows of FIG. 6 indicating the next scout region) to maximize the confidence. New scout regions 400 are scanned until the detection confidence is above a threshold. For example, each scout scan may be a single radial spoke or a narrow radial blade. The DRL AI is trained to predict the orientation of the next spoke or blade as well as its location.

A discounted reward for the policy or actor may minimize the time required to compute the diagnosis scan plan or detect the anatomy. In training, the AI policy is trained to optimize the time to detect the anatomy.

The AI operates as a single level of planning to reach the final objective of detecting the target anatomy. In another implementation, the DRL AI operates in a hierarchy or multiple levels. For example, the DRL AI selects a short-term plan, and each short-term plan indicates how to select a next of the different regions. The short-term plan may be an AI, such as DRL AI. The AI system is multiple levels for long and short-term planning. The long-term planning AI is trained to decide which short-term plan to follow next to detect the target anatomy, and each short-term planning system is trained to predict the next scout scan to acquire to achieve its own objective. The various short-term plans can differ by: target structure (e.g., identify highly visible and regular structures or landmarks (e.g., the bladder and femoral heads for a prostate scan) before attempting to identify the target structure); target accuracy (e.g., estimated localization uncertainty required to be achieved before moving to the next plan); scout scan properties (e.g., scan time, resolution, contrast, slice thickness, . . . ), and/or action space (e.g., size of spatial jumps between scout scans, allowed contrast changes, . . . ).

The DRL AI system may detect that patient positioning should be corrected to place the target anatomy within the scanning volume and that this correction should be performed by human intervention. In that case, the DRL AI system provides feedback to the user, such as a device technologist or the patient himself. The feedback describes how to correct patient positioning. Possible feedback channels include natural language instructions in spoken or written form, still or animated visual instructions displayed on a screen, haptic feedback through vibration transducers connected to the patient support subsystem, or another communication.

In act 230, the MR scanner images the target. The detected location of the target is used for diagnostic scanning. The MR scanner is configured by the position of the target for the imaging. The target is diagnostically imaged based on the final detected position of the target.

Once the target is detected, the processor configures the MR scanner to image the target. The configuration is provided as values of settings for the MR scanner, such as generating settings (values) for the RF subsystem, a gradient subsystem, and/or patient support subsystem (e.g., configuration parameters 340). The patient is physically positioned by the bed, such as moving the target to be within the FoV of the MR scanner. The patient position is adjusted relative to the MR scanner based on the detected target position. The RF pulses and gradients are then configured to diagnostically scan the target location. The localization of the target is used to configure the MR scanner to diagnostically scan the target. The target location is used to compute a plan for the diagnosis scan. The scan plan is converted into control parameters for the RF, gradient and mechanical actuators based on a model of the MR scanner (e.g., software to configure the MR scanner). The diagnosis scan is then executed by the MR scanner.

In one example, control parameters are computed to perform the diagnosis scan according to the scan plan. The components to control include the RF subsystem, the gradient subsystem, and the mechanical actuators for the patient support subsystem with respect to the scanning volume. The amount of required mechanical control may vary depending on the application and patient. If the target anatomy can fit within the scanning volume, then initial mechanical positioning is performed, and the diagnosis scan is performed using only RF and gradient controls. If the target anatomy is too large to fit within the homogeneous scanning volume, then mechanical adjustments are performed during the diagnosis scans to partially scan a target anatomy and then partially scan a remaining part of the target anatomy. The scan data or reconstructed images from the different diagnostic scans with the patient positioned differently relative to the MR scanner are concatenated, such as concatenating the partial images into a larger image of the whole target anatomy.

In a further interleaving approach, at least one of the scout scans is performed after beginning the diagnostic imaging. This interleaved scout scan is used to adjust for motion. The scan data or an image reconstructed from the scan data of the interleaved scout scan is used to refine the target position or correct the target position for motion. Additional scout scans may be acquired during the diagnostic scan to monitor patient motion, verify that the target anatomy is still at the same location, and perform adjustments to the scan plan if needed. The additional scout scans use a patient motion model and/or AI that predicts where the target anatomy is going to be at a future time. In some examples, the motion model assumes no motion by default, each additional scout scan is performed at the same location as the previous diagnostic scan, and the location of next diagnostic scan is computed using the additional scout scan. In other examples, the motion model incorporates slow motion due to physiological phenomena (e.g., displacement of the prostate due to bladder filling over the duration of a scan session). In that case, each consecutive scan is acquired at a different location form the previous scan according to the prior motion model. Each additional scout scan may be used to estimate parameters of the motion model (e.g., fit the model to the movement of the target) as well as adjust the location of the next diagnostic scan.

For the diagnostic scan, the MR scanner scans the patient. The scan is guided by a protocol, which establishes values for settings or control of the scanning. The values of the settings of the protocol are based, at least in part, on the location of the target anatomy. A pulse sequence (i.e., plurality of pulses from one or more coils) is created based on the configuration of the MR scanner (e.g., the imaging protocol selected). The pulse sequence is transmitted from coils into the patient. The resulting responses are measured by receiving radio frequency signals at the same or different coils. The scanning results in raw measurements as the scan data.

The protocol is for a medical test. The protocol is designed to provide scan data that may be used to reach a clinical finding. The scan data may be used to diagnose or answer a diagnostic question, such as whether more detailed scanning is needed, whether cancer exists in the organ, or a stage of cancer. The patient is referred to MR scanning for the clinical finding. For a given MR examination, the patient is positioned, the scan localized, and then the patient is scanned to find the clinical finding. The MR scanning continues over a period of seconds or minutes to acquire the scan data to answer the diagnostic questions. A diagnostic MR image may be generated for analysis by a physician (e.g., radiologist).

The MR scanning may use an image caliber MR scanner. In other embodiments, an MR scanner with a non-uniform main magnetic field, non-homogeneous pulses, and/or non-linear gradients is used to scan.

For simplicity, FIGS. 4, 5, and 6 show two-dimensional (2D) scans and 2D images. The approaches and examples apply as well for three-dimensional (3D) imaging. For 3D, the disk-shaped scan volumes shown in FIGS. 4, 5, and 6 are 3D spheres or 3D spherical shells. Other shapes than circular or spherical may be used.

The examples herein are for MR imaging. The use of multiple, separate scout scans may be used in other imaging modalities, such as computed tomography (CT) or X-ray imaging with smaller X-ray detectors, ultrasound scanners, or nuclear imaging scanners.

Below are illustrative examples. Examples of different of types (method, system, and non-transitory computer readable medium) may be used in other types. Different combinations of the examples may be provided.

In illustrative example 1, a method of localizing a target for magnetic resonance (MR) scanning comprises: estimating an initial position of the target relative to a magnetic resonance scanner from first data, the target comprising an object in a patient, and the first data comprises pre-scan information; performing multiple scout scans for different regions of the patient, the different regions based on the initial position; determining a subsequent position of the target relative to the magnetic resonance scanner from the scout scans; and diagnostically imaging the target with the magnetic resonance scanner, the imaging configured by the subsequent position of the target.

In illustrative example 2, the method of example 1, wherein the magnetic resonance scanner comprises a field of view with a diameter of spherical homogenous volume of less than 50 centimeters, wherein the scout scans are of different regions of the patient using the field of view.

In illustrative example 3, the method of example 2, wherein the diameter is less than 30 cm.

In illustrative example 4, the method of example 2, wherein the diameter is less than 10 cm.

In illustrative example 5, the method of any of examples 1-4, wherein estimating comprises estimating from the pre-scan information comprising patient registration data, measurements from an exterior of the patient, and/or a previously acquired representation of an interior of the patient.

In illustrative example 6, the method of any of examples 1-5, wherein estimating comprises estimating by an artificial intelligence in response to input of the pre-scan information.

In illustrative example 7, the method of any of examples 1-6, wherein performing comprises mechanically moving the patient relative to the magnetic resonance scanner for the scout scans of the different regions.

In illustrative example 8, the method of any of examples 1-7, wherein performing comprises adjusting a central frequency so that at least some of the different regions of the scout scans are outside of a homogenous scanning volume.

In illustrative example 9, the method of any of examples 1-8, wherein determining comprises aggregating scout scan data from the scout scans and estimating the subsequent position from the aggregated scout scan data.

In illustrative example 10, the method of any of examples 1-9, wherein performing and determining are interleaved so that one or more of the different regions are selected by a deep reinforcement learning artificial intelligence based on previous ones of the scout scans.

In illustrative example 11, the method of example 10, wherein the deep reinforcement learning artificial intelligence selects a short-term plan, and each short-term plan indicates how to select a next of the different regions.

In illustrative example 12, The method of any of examples 1-11, wherein diagnostically imaging comprises generating settings for radio frequency subsystem and a gradient subsystem of the magnetic resonance scanner to scan the subsequent location.

In illustrative example 13, the method of any of examples 1-12, wherein at least one of the scout scans is performed after beginning the diagnostically imaging, and further comprising adjusting for motion based on scan data of the at least one of the scout scans performed after beginning the diagnostically imaging.

In illustrative example 14, the method of any of examples 1-13, wherein diagnostically imaging comprises adjusting a patient position relative to the magnetic resonance scanner based on the subsequent position.

In illustrative example 15, a magnetic resonance (MR) system comprises a MR scanner configured by settings of controls to scan a diagnostic region of a patient, the scan providing scan data, the diagnostic region being within a homogenous volume field of view of the MR scanner; and a processor configured to scout scan different scout regions of the patient using values of the settings, configured to determine a location of the diagnostic region from the scout scan of the different scout regions, and configured to perform the scan of the diagnostic region based on the determined location.

In illustrative example 16, the MR system of example 15, wherein the values of the settings for the scout scan of the different scout regions use different contrast weightings for the different scout regions.

In illustrative example 17, the MR system of any of examples 15-16, wherein the processor is configured to estimate an initial location of the diagnostic region from a previous whole-body scan, the different scout regions established based on the initial location.

In illustrative example 18, The MR system of any of examples 15-17, further comprising: a patient support for the patient, the patient support moveable relative to the MR scanner; wherein the processor is configured to perform the scout scan and/or the scan of the diagnostic region with controlled movement of the patient support.

In illustrative example 19, the MR system of any of examples 15-18, wherein the processor is configured to scout scan and/or determine the location with an artificial intelligence previously trained to extrapolate information outside of a current field of view.

In illustrative example 20, a method for localizing a target for magnetic resonance (MR) scanning comprises: performing, by a magnetic resonance system, multiple scout scans of different regions of the patient, the magnetic resonance system having a field of view corresponding to a diameter of a spherical homogenous volume of 30 cm or less such that each of the different regions are outside the field of view of others of the different regions; determining, by a machine-learned model implemented by a processor, a position of the target relative to the magnetic resonance system from the multiple scout scans; and imaging the target with the magnetic resonance system, the imaging configured by the position of the target.

Although the subject matter has been described in terms of exemplary embodiments, it is not limited thereto. Rather, the appended claims should be construed broadly, to include other variants and embodiments, which can be made by those skilled in the art.

What is claimed is:

1. A method of localizing a target for magnetic resonance (MR) scanning, the method comprising:
estimating an initial position of the target relative to a magnetic resonance scanner from first data, the target comprising an object in a patient, and the first data comprises pre-scan information;
performing multiple scout scans for different regions of the patient, the different regions based on the initial position;
determining a subsequent position of the target relative to the magnetic resonance scanner from the scout scans; and
diagnostically imaging the target with the magnetic resonance scanner, the imaging configured by the subsequent position of the target.

2. The method of claim 1, wherein the magnetic resonance scanner comprises a field of view with a diameter of spherical homogenous volume of less than 50 centimeters, wherein the scout scans are of different regions of the patient using the field of view.

3. The method of claim 2, wherein the diameter is less than 30 cm.

4. The method of claim 2, wherein the diameter is less than 10 cm.

5. The method of claim 1, wherein estimating comprises estimating from the pre-scan information comprising patient registration data, measurements from an exterior of the patient, and/or a previously acquired representation of an interior of the patient.

6. The method of claim 1, wherein estimating comprises estimating by an artificial intelligence in response to input of the pre-scan information.

7. The method of claim 1, wherein performing comprises mechanically moving the patient relative to the magnetic resonance scanner for the scout scans of the different regions.

8. The method of claim 1, wherein performing comprises adjusting a central frequency so that at least some of the different regions of the scout scans are outside of a homogenous scanning volume.

9. The method of claim 1, wherein determining comprises aggregating scout scan data from the scout scans and estimating the subsequent position from the aggregated scout scan data.

10. The method of claim 1, wherein performing and determining are interleaved so that one or more of the different regions are selected by a deep reinforcement learning artificial intelligence based on previous ones of the scout scans.

11. The method of claim 10, wherein the deep reinforcement learning artificial intelligence selects a short-term plan, and each short-term plan indicates how to select a next of the different regions.

12. The method of claim 1, wherein diagnostically imaging comprises generating settings for radio frequency subsystem and a gradient subsystem of the magnetic resonance scanner to scan the subsequent location.

13. The method of claim 1, wherein at least one of the scout scans is performed after beginning the diagnostically imaging, and further comprising adjusting for motion based on scan data of the at least one of the scout scans performed after beginning the diagnostically imaging.

14. The method of claim 1, wherein diagnostically imaging comprises adjusting a patient position relative to the magnetic resonance scanner based on the subsequent position.

15. A magnetic resonance (MR) system comprising:
a MR scanner configured by settings of controls to scan a diagnostic region of a patient, the scan providing scan data, the diagnostic region being within a homogenous volume field of view of the MR scanner; and
a processor configured to scout scan different scout regions of the patient using values of the settings, configured to determine a location of the diagnostic region from the scout scan of the different scout regions, and configured to perform the scan of the diagnostic region based on the determined location.

16. The MR system of claim 15, wherein the values of the settings for the scout scan of the different scout regions use different contrast weightings for the different scout regions.

17. The MR system of claim 15, wherein the processor is configured to estimate an initial location of the diagnostic region from a previous whole-body scan, the different scout regions established based on the initial location.

18. The MR system of claim 15, further comprising:
a patient support for the patient, the patient support moveable relative to the MR scanner;
wherein the processor is configured to perform the scout scan and/or the scan of the diagnostic region with controlled movement of the patient support.

19. The MR system of claim 15, wherein the processor is configured to scout scan and/or determine the location with an artificial intelligence previously trained to extrapolate information outside of a current field of view.

20. A method for localizing a target for magnetic resonance (MR) scanning, the method comprising:
performing, by a magnetic resonance system, multiple scout scans of different regions of the patient, the magnetic resonance system having a field of view corresponding to a diameter of a spherical homogenous volume of 30 cm or less such that each of the different regions are outside the field of view of others of the different regions;
determining, by a machine-learned model implemented by a processor, a position of the target relative to the magnetic resonance system from the multiple scout scans; and
imaging the target with the magnetic resonance system, the imaging configured by the position of the target.
